(19) United States
(12) Patent Application Publication
(10) Pub. No.: US 2022/0202832 A1
(43) Pub. Date: Jun. 30, 2022

(54) ALLOPREGNANOLONE COMPOSITIONS AND USES THEREOF

(71) Applicant: The Regents of the University of California, Oakland, CA (US)

(72) Inventors: Michael A. ROGAWSKI, Sacramento, CA (US); Dorota ZOLKOWSKA, Sacramento, CA (US)

(21) Appl. No.: 17/601,705

(22) PCT Filed: Apr. 6, 2020
(86) PCT No.: PCT/US2020/026955

§ 371 Date: Oct. 5, 2021

Related U.S. Application Data
(60) Provisional application No. 62/830,012, filed on Apr. 5, 2019.
(60) Provisional application No. 62/831,877, filed on Apr. 10, 2019.

(51) Int. Cl. A61K 31/573; A61K 47/69; A61K 9/00; A61K 31/5517; A61K 31/5513; A61P 25/08; A61P 25/14; A61P 25/28

(52) U.S. Cl. CPC A61K 31/573; A61K 47/6951; A61K 9/0043; A61P 25/28; A61K 31/5513; A61P 25/08; A61P 25/14; A61K 31/5517

(57) ABSTRACT

This disclosure is related to non-invasive methods of treating, preventing, inhibiting, delaying, and/or mitigating conditions that are modulated by physiological levels of allopregnanolone and are susceptible to allopregnanolone therapy.

### STATEMENT OF GOVERNMENTAL SUPPORT

This invention was made with Government support under Grant Number 1U54NS079202 awarded by the National Institutes of Health/National Institute of Neurological Disorders and Stroke (NIH/NINDS). The Government has certain rights in the invention.

### FIELD

The present disclosure relates to methods of treating and/or preventing conditions that are modulated by physiological levels of allopregnanolone and/or are susceptible to allopregnanolone therapy.

### BACKGROUND

Allopregnanolone is a neurosteroid implicated in many disease states. Neuroactive steroids such as allopregnanolone alter neuronal excitability through interactions with specific neurotransmitter receptors. Allopregnanolone is a potent positive allosteric modulator of the GABA(A) receptor complex. GABAergic mechanisms are implicated in depression and anxiety. Reduced levels of allopregnanolone in the peripheral blood or cerebrospinal fluid were found to be associated with major depression, anxiety disorders, premenstrual dysphoric disorder, negative symptoms in schizophrenia, or impulsive aggression. Schille C. et al. Prog Neurobiol. 2014; 113:79-87. Bäckström T. et al., Prog Neurobiol. 2014; 113:88-94, have described the role of allopregnanolone in mood disorders. Irwin R. W., et al. Front. Cell. Neurosci., 30 Jul. 2014, have described therapeutic efficacy of allopregnanolone in multiple neurological diseases including Alzheimer's disease, Parkinson's disease, multiple sclerosis, Niemann-Pick, diabetic neuropathy, status epilepticus, and traumatic brain injury. Noorbakhsh F. et al. Front. Cell. Neurosci., 3 Jun. 2014, have described the role of allopregnanolone in neuroinflammation. Human clinical trials have demonstrated the efficacy of intravenous allopregnanolone in the treatment of postpartum depression. Kanes et al., (2017) Human Psychopharmacology, Volume 32, Issue 232:e2576, and Kanes et al., Lancet 2017; 390: 480-89.

Despite the known therapeutic benefits of allopregnanolone, pharmaceutical product development has been hindered by the physico-chemical properties of allopregnanolone. Irwin, R. W., et al., Frontiers in Cellular Neuroscience, 2014, 8, pp 1-19 report a log P value for allopregnanolone of about 5.042, which poses a solubility challenge for dissolution of the compound in aqueous biological fluids and for enteral absorption. In addition, it has been found experimentally that allopregnanolone is not orally bioavailable even when solubilized with excipients such as sulfobutyl ether β-cyclodextrin, sodium salt (e.g., CAPTISOL®). Irwin R. W. et al., PLoS One. 2015 Jun. 3; 10(6):e0128313 tested several possible routes of delivery for allopregnanolone and concluded that intramuscular delivery was optimal. To avoid the problems with allopregnanolone absorption in vivo, allopregnanolone compositions have typically been developed for parenteral routes of administration. But parenteral administration is invasive, causes patient discomfort, and reduces patient compliance. Allopregnanolone has a high clearance so that concentrations in the blood fall rapidly. In mice, it has been found that the plasma concentration half-time (t_{1/2}) was 16 min (Zolkowska D. et al., Epilepsia 2018 October; 59 Suppl 2:220-227), whereas in a human the t_{1/2} was 35 min (Wang Neurotherapeutics 2017 October; 14(4): 1073-1083). As allopregnanolone is not orally bioavailable, it has been administered using a prolonged continuous intravenous infusion for the treatment of human disease. Meltzer-Brody S. et al. Lancet 2018; 392:1058-1070. There is a need in the field for non-parenteral modes of administration of allopregnanolone that can deliver therapeutically relevant amounts of allopregnanolone to target sites in the body over a prolonged duration of time following dosing.

### SUMMARY

Provided herein are non-invasive methods of treating, preventing, inhibiting, delaying, and/or mitigating conditions that are modulated by physiological levels and/or fluctuations in phmysiological levels of allopregnanolone and/or are susceptible to allopregnanolone therapy. The methods provide sustained, therapeutic blood levels of allopregnanolone over many hours. The methods also deliver allopregnanolone directly into the brain to target brain areas associated with beneficial therapeutic effects while reducing or avoiding delivery to other brain regions associated with adverse effects.

In one aspect, provided herein is a method of treating or preventing a condition susceptible to allopregnanolone selected from the group consisting of traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, and smoking cessation, in a subject in need thereof, comprising administering to the subject intranasally, buccally, sublingually, lingually or transdermally, a composition comprising allopregnanolone and a pharmaceutically acceptable excipient suitable for solubilizing allopregnanolone.

Also provided herein are methods and compositions for use as a component of moderate sedation/analgesia (conscious sedation) to allay anxiety and reduce or limit pain in patients in need thereof for diagnostic or therapeutic applications.

Further provided herein is a method for selective delivery of allopregnanolone to the olfactory bulb and adjacent structures, the method comprising intranasally administering a composition comprising allopregnanolone and sulfobutylether-β-cyclodextrin, wherein, after intranasally administering, the tissue concentration of allopregnanolone in the olfactory blub and adjacent brain structures is higher compared to the tissue concentration in other regions of the brain or in the whole brain.

Also described herein is the use of an intranasal, buccal, sublingual, lingual or transdermal composition of allopregnanolone for treating or preventing any condition susceptible to allopregnanolone described herein.

### BRIEF DESCRIPTION OF THE DRAWINGS

FIG. 1 shows the effect of intranasal allopregnanolone on the seizure threshold in the timed intravenous pentylenetetrazol (PTZ) seizure threshold test at 10 minutes, 1 hours and 8 hours.

FIG. 2 shows percent seizure protection in 6 Hz mode over time after intranasal administration of allopregnanolone (16 mg/kg) in mice as described in the Examples section.

FIG. 3 shows the plasma levels of allopregnanolone over time after intranasal or intramuscular administration of allopregnanolone as described in the Examples section.

FIG. 4 shows the percent of animals exhibiting loss of righting reflex (motor impairment) after intramuscular or intranasal administration of allopregnanolone (16 mg/kg) or vehicle in mice as described in the Examples section.

FIG. 5 shows analysis of allopregnanolone concentrations in various brain regions (sections of the mouse brain) after intranasal dosing.

FIG. 6 shows results of intravenous PTZ threshold tests to assess the effect of intranasal administration of allopregnanolone in mice.

FIG. 7 shows the results of PTZ seizure threshold tests to assess the effect of intranasal administration of allopregnanolone in mice.

FIG. 8 shows the results of an intravenous Bicuculline Threshold Test for animals dosed with intranasal allopregnanolone.

FIG. 9 shows the effect of intranasal administration of allopregnanolone, midazolam, and diazepam as measured using the horizontal screen test to assess motor impairment.

FIG. 10 shows levels of impairment as measured using the loss of righting reflex test following intranasal allopregnanolone administration.

FIG. 11 depicts the results of pharmacokinetic studied measuring allopregnanolone levels in plasma and in various brain regions following intranasal dosing.

### DETAILED DESCRIPTION

Described herein are allopregnanolone compositions which are suitable for non-parenteral routes of administration (e.g., intranasal, buccal, sublingual, lingual or transdermal administration) and provide improved pharmacokinetic and pharmacodynamic properties to achieve prolonged (many hours) systemic and brain exposure and an optimal balance between therapeutic and side effect profiles in mammals. The compositions of allopregnanolone described herein provide a route for allopregnanolone delivery that can potentially circumvent poor gastrointestinal bioavailability, potentially bypass the blood-brain barrier, and deliver therapeutically meaningful levels of allopregnanolone to the brain, while reducing potential side effects associated with systemic exposure to allopregnanolone. It was surprisingly found that intranasal administration of allopregnanolone compositions described herein led to a longer period of efficacy in an animal model, when compared with parenteral (e.g., intramuscular) administration of allopregnanolone compositions. It was further surprisingly found that intranasal administration of allopregnanolone compositions described herein led to selective increased exposure of allopregnanolone in brain regions relevant to the therapeutic activity compared with the exposure in brain regions relevant to adverse-effects or toxicity.

It was further found that intranasal allopregnanolone raises PTZ and bicuculline seizure thresholds and prolongs the time to occurrence of maximal PTZ-induced seizures. Thus, intranasal delivery is a feasible route of administration for allopregnanolone and can overcome the poor oral bioavailability of the neuroactive steroid.

It is recognized that with nasal delivery, a large percentage of the dose may still be transported into the gastrointestinal tract by mucociliary clearance. Ingested allopregnanolone would have only minimal bioactivity but could reduce the bioavailability following intranasal administration. A surprising discovery of the current disclosure is the pharmacokinetic results described herein that suggest that little is lost by this route. The dose adjusted Cₘₐₓ for intramuscular allopregnanolone in a prior study is 2150 ng/ml (Zolkowska et al., 2018). This contrasts with the Cₘₐₓ in the present study of 3500 ng/ml. Thus, if anything, intranasal administration may be superior to other forms of parenteral administration.

Additionally, based on the studies described herein, intranasal allopregnanolone fails to cause substantial sedation or motor impairment at antiseizure doses. This is in contrast to the benzodiazepines midazolam and diazepam, which dramatically impair motor function in the horizontal screen test at doses that have comparable antiseizure activity. In addition, intranasal allopregnanolone causes only minimal loss of righting reflex whereas the same dose administered intramuscularly causes profound sedation as assessed by this test.

As disclosed herein, intranasal allopregnanolone acts rapidly, within <5 min and possibly faster. This contrasts with a Tₘₐₓ of 60 min when allopregnanolone is administered intramuscularly (Zolkowska et al., 2018). It is noteworthy that other steroids including antiinflammatory steroids are commonly administered intranasally, such as for the treatment of allergic rhinitis. Such steroids are intended to act locally and commonly used agents such as fluticasone propionate have negligible systemic availability, although older agents such as flunisolide have substantial systemic availability (Lumry, 1999). Prior studies with intranasal progesterone, which does have systemic availability, has demonstrated rapid absorption (<2 min) as is the case for allopregnanolone (Cicinelli et al., 1991). The speed of onset implies that, in some embodiments, intransal allopregnanolone can be used to treat certain acute indications, such as acute seizures.

As shown in studies described herein, following intranasal administration, allopregnanolone levels are present at high levels in the olfactory bulb. These levels are substantially higher than the plasma levels. This contrasts with the other brain regions where the levels are lower or equivalent to the plasma levels.

The methods described herein allow for allopregnanolone's therapeutic effects to last for durations of several hours, thereby making it practical to administer the treatment only a few times a day. The methods described herein simulate the prolonged allopregnanolone exposure obtained with a therapeutically relevant intravenous infusion that is carried out over a prolonged duration such as 60 hours. Non-invasive delivery methods are desirable because they improve patient comfort and the patient can easily self-administer the treatment or a lay caregiver can administer the treatment without the need for specialized healthcare personnel or confinement to a specialized facility.

### Definitions

As used herein, the term “about,” when qualifying a number, e.g., about 15% w/w, refers to the number qualified and optionally the numbers included in a range about that qualified number that includes ±10% of the number. Thus compositions slightly outside the cited ranges are also encompassed by the scope of embodiments presented herein.

As used herein, “administering” refers to local and/or systemic administration. Parenteral administration includes, e.g., intravenous, intramuscular, intra-arterial, intradermal, subcutaneous, intraperitoneal, or intraventricular administration. Buccal administration refers to administration between the gums and cheeks (e.g., by injection, spraying, painting of a liquid composition between the gums and cheeks, or by use of a fast-dissolving tablet/lozenge/strip). Sublingual or lingual administration refers to administration in the mucosa below the tongue or near the tongue (e.g., by injection, spraying, painting of a liquid composition below the tongue or near the tongue, or by use of a fast-dissolving lingual or sublingual tablet/lozenge/strip). Intranasal administration refers to administration in the nasal cavity (e.g., by injection, spraying, or painting of a liquid composition in the nasal cavity). Transdermal administration refers to administration across the skin (e.g., by using a patch).

The term “effective amount” or “pharmaceutically effective amount” or “therapeutically effective amount” refers to the amount and/or dosage, and/or dosage regime of allopregnanolone necessary to bring about the desired result e.g., an amount sufficient prevent, abort or terminate a seizure, or lessen the symptoms of depression.

As used herein, the terms “treating” and “treatment” refer to delaying the onset of, retarding or reversing the progress of, reducing the severity of, or alleviating or preventing either the disease or condition to which the term applies, or one or more symptoms of such disease or condition. “Preventing” a disease or condition refers to prophylactic administration of a composition described herein to reduce symptoms and/or delay the onset of symptoms and/or inhibit the onset of symptoms.

The terms “reduce,” “delay,” “relieve,” “alleviate” refer to the detectable decrease in the frequency, severity and/or duration of symptoms (e.g., seizures, symptoms of depression). A reduction in the frequency, severity and/or duration of symptoms may be measured by self-assessment (e.g., by reporting of the patient) or by a trained clinical observer. Any reduction of the frequency, severity and/or duration of symptoms is determined by comparing patient status before and after treatment.

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other healthworker in a hospital, psychiatric care facility, as an outpatient, or other clinical context. In certain embodiments the subject may not be under the care or prescription of a physician or other healthworker.

“Condition susceptible to allopregnanolone” refers to a condition that is modulated by physiological levels of allopregnanolone and/or fluctuations in physiological levels of allopregnanolone, and consequently susceptible to allopregnanolone therapy. In some cases, reduced physiological levels of allopregnanolone may underlie the condition or the symptoms of the condition. In some cases, allopregnanolone may be an allosteric modulator for other targets (e.g., GABAergic receptor) and reduced physiological levels of allopregnanolone may underlie the condition or the symptoms of the condition.

“Allopregnanolone,” also known as 3α-hydroxy-5α-pregnan-20-one or 3α,5α-tetrahydroprogesterone, IUPAC name 1-(3-Hydroxy-10,13-dimethyl-2,3,4,5,6,7,8,9,11,12,14,15,16,17-tetradecahydro-1H-cyclopenta[a]phenanthren-17-yl)ethanone, and referenced as CAS number 516-54-1, is a metabolite of progesterone. The chemical structure of allopregnanolone is depicted below in Formula I:

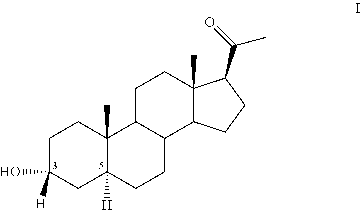 [SMILES: CC(=O)[C@H]1CCC2C3CC[C@H]4C[C@H](O)CC[C@]4(C)C3CC[C@@]21C]

In various embodiments, the compositions described herein may comprise one or more salts of allopregnanolone, e.g., a sulfate, a phosphate, a mesylate, a hemisuccinate, and the like. In some embodiments, the compositions described herein may comprise one or more derivatives of allopregnanolone, e.g., nitrosylated derivatives.

Other neurosteroids that find use in the present compositions and methods include without limitation allotetrahydrodeoxycorticosterone (3α,21-dihydroxy-5α-pregnan-20-one; THDOC), 3 α,21-dihydroxy-5β-pregnan-20-one, pregnanolone (3α-hydroxy-5P-pregnan-20-one), Ganaxolone (INN, also known as CCD-1042; IUPAC name (3α,5α)-3-hydroxy-5-methylpregnan-20-one; 1-[(3R,5S,8R,9S,10S,13S,14S; 17 S)-3-hydroxy-3, 10, 13-trimethyl-1,2,4,5,6,7,8,9,11,12,14,15,16,17-tetradecahydrocyclopenta[a]phenanthren-17-yl]ethanone), alphaxo one; alphadolone, hydroxydione, minaxolone, and Althesin (a mixture of alphaxolone, alphadolone, tetrahydrodeoxycorticosterone, pregnenolone, dehydroepiandrosterone (DHEA), 7-substituted benz[e]indene-3-carbonitriles (see, e.g., Hu, et al, J Med Chem. (1993) 36(24):3956-67); 7-(2-hydroxyethyl)benz[e]indene analogues (see, e.g., Han, et al, J Med Chem. (1995) 38(22):4548-56); 3α-hydroxy-5α-pregnan-20-one and 3α-hydroxy-5 P-pregnan-20-one analogues (see, e.g., Han, et al, J Med Chem. (1996) 39(21):4218-32); enantiomers of dehydroepiandrosterone sulfate, pregnenolone sulfate, and (3 α,5P)-3-hydroxypregnan-20-one sulfate (see, e.g., Nilsson, et al, J Med Chem. (1998) 41(14):2604-13); 13,24-cyclo-18,21-dinorcholane analogues (see, e.g., Jiang, et al, J Med Chem. (2003) 46(25):5334-48), N-acylated 17a-aza-D-homosteroid analogues (see, e.g., Covey, et al., J Med Chem. (2000) 43(17):3201-4); 5 p-methyl-3-ketosteroid analogues (see, e.g., Zeng, et al., J Org Chem. (2000) 65(7):2264-6); 18-norandrostan-17-one analogues (see, e.g., Jiang, et al., J Org Chem. (2000) 65(11):3555-7); (3α,5α)- and (3 α,5P)-3-hydroxypregnan-20-one analogs (see, e.g., Zeng, et al, J Med Chem. (2005) 48(8):3051-9); benz[f]indenes (see, e.g., Scaglione, et al, J Med Chem. (2006) 49(15):4595-605); enantiomers of androgens (see, e.g.; Katona, et al, Eur J Med Chem. (2008) 43(1): 107-13); cyclopenta[b]phenanthrenes and cyclopenta[b]anthracenes (see, e.g., Scaglione, et al, J Med Chem. (2008) 51(5): 1309-18); 2P-hydroxygonane derivatives (see, e.g., Wang, et al, Tetrahedron (2007) 63(33):7977-7984); Δ¹⁶-alphaxalone and corresponding 17-carbonitrile analogues (see, e.g., Bandyopadhyaya, et at, Bioorg Med Chem Lett. (2010) 20(22):6680-4); A(16) and Δ(17(20)) analogues of A(16)-alphaxalone (see, e.g., Stastna, et at, J Med Chem. (2011) 54(11)3926-34); neurosteroid analogs developed by CoCensys (now Purdue Neuroscience) (e.g., CCD-3693, Co2-6749 (a.k.a., GMA-839 and WAY-141839) or Sage Therapeutics (e.g., SAGE-217 (CAS No.: 1632051-40-1; 3α-Hydroxy-3β-methyl-21-(4-cyano-4H-pyrazol-1′-yl)-19-nor-5β-pregnan-20-one; 3β-Methyl-21-(4-cyano-1H-pyrazol-1′-yl)-19-norpregnanolone; 3α-Hydroxy-3β-methyl-5β-dihydro-21-(4-cyano-1H-pyrazol-V-yl)-19-norprogesterone), SAGE-547 (brexanolone); SAGE-324); neurosteroid analogs described in U.S. Pat. No. 7,781,421; U.S. Patent Publication Nos. US2015/0158903 and US2015/0175651; and in PCT Patent Publications WO2008/157460; WO 1993/003732; WO 1993/018053; WO 1994/027608; WO 1995/021617; WO 1996/016076; WO 1996/040043; WO/2018/039378; WO/2018/039378; WO/2018/013613; WO/2018/013615; WO/2018/009867; WO/2017/193046; WO/2017/173358; WO/2017/156103; WO/2017/087864; WO/2017/007832; WO/2017/007836; WO/2017/007840; WO/2016/205721; WO/2016/164763; WO/2016/134301; WO/2016/123056; WO/2016/082789; WO/2016/061537; WO/2016/061527; WO/2016/057713; WO/2016/040322; WO/2015/195967; WO/2015/195962; WO/2015/180679; WO/2015/027227; WO/2015/010054; WO/2014/169832; WO/2014/169831; WO/2014/169836; WO/2014/169833; WO/2014/160480; WO/2014/160441; WO/2014/100228; WO/2013/188792; WO/2013/112605; WO/2013/056181; and WO/2013/036835, as well as salts, hemi succinates, sulfates and/or nitrosylated derivatives thereof. The foregoing listed patents and patent publications are hereby incorporated herein by reference in their entireties for all purposes.

In one aspect, provided herein is a method of treating or preventing a condition susceptible to allopregnanolone. In certain embodiments, the condition is selected from the group consisting of traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, and smoking cessation, in a subject in need thereof. The methods comprise administering to a subject in need thereof an intranasal, buccal, sublingual lingual or transdermal composition comprising allopregnanolone and a pharmaceutically acceptable excipient suitable for solubilizing allopregnanolone.

In some embodiments, the allopregnanolone compositions described herein are administered bucally. In some embodiments, the allopregnanolone compositions described herein are administered sublingually In some embodiments, the allopregnanolone compositions described herein are administered lingually. In some embodiments, the allopregnanolone compositions described herein are administered transdermally. In some embodiments, the allopregnanolone compositions described herein are administered intranasally.

In a group of embodiments, the pharmaceutically acceptable excipient suitable for solubilizing allopregnanolone is β-cyclodextrin, hydroxypropyl-β-cyclodextrin or sulfobutylether-β-cyclodextrin, or a combination thereof. In some embodiments, the pharmaceutically acceptable excipient suitable for solubilizing allopregnanolone is β-cyclodextrin. In some embodiments, the pharmaceutically acceptable excipient is hydroxypropyl-β-cyclodextrin. In some embodiments, the pharmaceutically acceptable excipient suitable for solubilizing allopregnanolone is sulfobutylether-β-cyclodextrin.

In one aspect, provided herein is a method of treating or preventing a condition susceptible to allopregnanolone selected from the group consisting of traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, and smoking cessation, in a subject in need thereof, comprising administering to the subject an intranasal composition comprising allopregnanolone and sulfobutylether-β-cyclodextrin.

In a group of embodiments, the condition susceptible to allopregnanolone is a mood disorder. In some instances, the mood disorder is anxiety (used interchangeably herein with “an anxiety disorder”). Examples of anxiety disorders include and are not limited to generalized anxiety disorder, panic disorder, social anxiety disorder, obsessive compulsive disorder, and post-traumatic stress disorder. In some cases, the mood disorder is depression. Examples of depression include and are not limited to PTSD, PPD, MDD, PDD, PMDD, bipolar disorder (cyclothymia), SAD or secondary depression. In a group of embodiments, the condition susceptible to allopregnanolone is anxiety, post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD) or secondary depression.

In one instance, the condition susceptible to allopregnanolone is postpartum depression (PPD). In another instance, the condition susceptible to allopregnanolone is post-traumatic stress disorder (PTSD). In further instances, the condition susceptible to allopregnanolone is major depressive disorder (MDD). In additional instances, the condition susceptible to allopregnanolone is secondary depression. In some instances, the condition susceptible to allopregnanolone is premenstrual dysphoric disorder (PMDD). In some instances, the condition susceptible to allopregnanolone is persistent depressive disorder (PDD). In some instances, the condition susceptible to allopregnanolone is seasonal affective disorder (SAD). In some instances, the condition susceptible to allopregnanolone is treatment resistant depression. In some instances, the condition susceptible to allopregnanolone is bipolar disorder. In some instances, the condition susceptible to allopregnanolone is mild bipolar disorder (cyclothymia). In some instances, the condition susceptible to allopregnanolone is secondary depression, e.g., depression that is secondary to a thyroid gland disorder, cancer, cardiovascular disease, or prolonged pain.

In some instances, the condition susceptible to allopregnanolone is epilepsy or seizures. In some of such instances, the seizures are acute seizures, tonic seizures, clonic seizures, or seizure clusters. In some instances, the epilepsy is status epilepticus or myoclonic epilepsy.

In some instances, the condition susceptible to allopregnanolone is a neurodegenerative condition. In some of such instances, the neurodegenerative condition susceptible to allopregnanolone is Alzheimer's disease or Parkinson's disease.

In any of the embodiments described herein, in one example, the sulfobutylether-β-cyclodextrin is sulfobutylether-β-cyclodextrin sodium salt. In some instances, the composition comprising allopregnanolone described herein comprises about 10-25 mg/mL of allopregnanolone in a solution of sulfobutylether-β-cyclodextrin sodium salt in saline. In some of such embodiments, the solution of sulfobutylether-β-cyclodextrin sodium salt in saline comprises about 10-60% sulfobutylether-β-cyclodextrin sodium salt in saline. In certain instances, the saline is about 0.9% saline. In some instances, the composition comprising allopregnanolone is an intranasal composition that comprises about 16 mg/mL allopregnanolone in about 40% sulfobutylether-β-cyclodextrin in about 0.9% saline.

In some embodiments, allopregnanolone and the pharmaceutically acceptable excipient (e.g., β-cyclodextrin, hydroxypropyl-β-cyclodextrin or sulfobutyl ether β-cyclodextrin, or a combination thereof) are formulated as an aqueous composition (e.g., in water or saline) comprising allopregnanolone at a concentration of about 0.25 mg/mL, about 0.5 mg/mL; about 1.0 mg/mL; about 1.5 mg/mL; about 2.0 mg/mL; about 2.5 mg/mL; about 3.0 mg/mL; about 3.5 mg/mL; about 4.0 mg/mL; about 4.5 mg/mL; about 5.0 mg/mL, about 5.5 mg/mL, about 6.0 mg/mL, about 6.5 mg/mL, about 7.0 mg/mL, about 7.5 mg/mL, about 8.0 mg/mL, about 8.5 mg/mL, about 9.0 mg/mL, about 9.5 mg/mL, about 10 mg/mL, about 15 mg/mL, about 20 mg/mL, about 25 mg/mL, or about 30 mg/mL. In other embodiments, allopregnanolone and the pharmaceutically acceptable excipient (e.g., β-cyclodextrin, hydroxypropyl-β-cyclodextrin or sulfobutyl ether β-cyclodextrin, or a combination thereof) are formulated as an aqueous composition (e.g., in water or saline) comprising allopregnanolone at a concentration of about 1.5 mg/mL. In other embodiments, allopregnanolone and the pharmaceutically acceptable excipient (e.g., β-cyclodextrin, hydroxypropyl-β-cyclodextrin or sulfobutyl ether β-cyclodextrin, or a combination thereof) are formulated as an aqueous composition (e.g., in water or saline) comprising allopregnanolone at a concentration of about 5 mg/mL. In other embodiments, allopregnanolone and the pharmaceutically acceptable excipient (e.g., β-cyclodextrin, hydroxypropyl-β-cyclodextrin or sulfobutyl ether β-cyclodextrin, or a combination thereof) are formulated as an aqueous composition (e.g., in water or saline) comprising allopregnanolone at a concentration of about 15 mg/mL.

In some or any embodiments described herein, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) comprising allopregnanolone at a concentration between about 5-400 mg/mL; about 5-300 mg/mL; about 5-250 mg/mL; about 25-300 mg/mL; about 25-200 mg/mL; about 25-100 mg/mL; about 25-50 mg/mL; about 50-400 mg/mL; about 50-300 mg/mL; about 60-400 mg/mL; about 60-300 mg/mL; about 150-400 mg/mL; about 150-300 mg/mL; about 200-300 mg/mL; about 200-400 mg/mL; about 30-100 mg/mL; about 300-400 mg/mL; about −100 mg/mL; about 45-75 mg/mL; about 50-70 mg/mL; about 55-65 mg/mL; or about 50-60 mg/mL. In other embodiments, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) comprising allopregnanolone at a concentration of about 5 mg/mL; about 10 mg/mL; about 15 mg/mL; about 20 mg/mL; about 25 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/mL; about 75 mg/mL; about 80 mg/mL; about 85 mg/mL; about 90 mg/mL, about 95 mg/mL; about 100 mg/mL; about 150 mg/mL; about 200 mg/mL; about 250 mg/mL; about 300 mg/mL; about 350 mg/mL; or about 400 mg/mL.

In other embodiments, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) comprising between about 2.5-60%, about 2.5-40%, about 2.5-30%, about 2.5-20%, about 2.5-10%, about 5-40%, about 5-30%, about 5-20%, about 5-10%, about 6-40%, about 6-30%, about 6-20%, about 6-10%, about 10-40%, about 10-30%, about 10-20%, about 20-40%, about 20-30%, about 25-40%, about 25-30%, about 3-10%, about 4.5-7.5%, about 5-7%, about 5.5-6.5% of the pharmaceutically acceptable excipient. In other embodiments, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) is formulated as an aqueous composition (e.g., in water or saline) comprising about 2.5%, about 3%, about 4%, about 4.5%, about 5%, about 5.5%, about 6%, about 6.5%, about 7%, about 7.5%, about 8%, about 8.5%, about 9%, about 9.5%, about 10%, about 15%, about 20%, about 25%, about 30%, about 35% or about 40% of the pharmaceutically acceptable excipient. In an embodiment, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) comprising about 5% of the pharmaceutically acceptable excipient. In other embodiments, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) comprising about 6% of the pharmaceutically acceptable excipient. In an embodiment, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) comprising about 10% of the pharmaceutically acceptable excipient. In an embodiment, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) comprising about 15% of the pharmaceutically acceptable excipient. In an embodiment, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) comprising about 20% of the pharmaceutically acceptable excipient. In an embodiment, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) comprising about 30% of the pharmaceutically acceptable excipient. In an embodiment, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) comprising about 40% of the pharmaceutically acceptable excipient. In some of such embodiments, the sulfobutyl ether β-cyclodextrin is sulfobutyl ether β-cyclodextrin, sodium salt (e.g., CAPTISOL®).

In any of the compositions described herein, the allopregnanolone composition is a sterile composition and may comprise from about 5 mg/mL to about 250 mg/mL of the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof).

In some embodiments, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) with a pH between about 3-10, about 4-9, about 4-8, about 4-7, about 4-6, about 4-5, about 5-9, about 5-8, about 5-7, about 5-6, about 4.5-7.5, or about 5.5-7.5. In some embodiments, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) with a pH of about 3, 3.5, 4, 4.5, 5, 5.5, 6, 6.5, 7, 7.5, 8, 8.5, or 9. In some embodiments, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) with a pH of about 5-8. In some embodiments, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) with a pH of about 5. In some embodiments, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) with a pH of about 6. In some embodiments, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) with a pH of about 7. In some embodiments, allopregnanolone and the pharmaceutically acceptable excipient suitable for solubilizing allopregnanolone (e.g., β-cyclodextrin, hydroxypropyl-β-cyclodextrin or sulfobutyl ether β-cyclodextrin, or a combination thereof) are formulated as an aqueous composition (e.g., in water or saline) with a pH of about 8. In some of such embodiments, the composition is buffered with a citrate buffer. In some other such embodiments, the composition is buffered with a phosphate buffer. Any suitable buffer may be used to achieve the desired pH and such buffers are known to a practitioner of skill.

In some embodiments, the allopregnanolone compositions described herein, depending on the dosage form, may further comprise pharmaceutically compatible carrier materials such as solvents (e.g., ethanol), oils (e.g., edible oils), binders, suspending agents, disintegration agents, filling agents, surfactants, solubilizers, stabilizers, lubricants, wetting agents, diluents, preservatives, and the like. “Pharmaceutically compatible carrier materials” include, and are not limited to, acacia, gelatin, colloidal silicon dioxide, calcium glycerophosphate, calcium lactate, maltodextrin, glycerin, magnesium silicate, polyvinylpyrrolidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, canola oil, sesame oil, peanut oil, and the like. See, e.g., Remington: The Science and Practice of Pharmacy, Twenty-Second Ed (Philadelphia, Pa.: Pharmaceutical Press, 2012); Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins 1999).

Nasal excipients that may be used include embodiments disclosed herein include, but are not limited to water soluble polymers, surfactants, co-solvents, chelating agents, stabilizing agents, isotonizing agents, buffer substances, preservatives, thickeners, complexing agents, permeation enhancers, flavor modifiers, and electrolytes.

Water soluble polymers that may be used are selected from but not limited to methylcellulose, hydroxyethyl cellulose, hydroxypropyl cellulose, hydroxypropyl methylcellulose, hydroxypropyl ethylcellulose, sodium carboxymethylcellulose, polyvinyl alcohol, polyvinylpyrrolidone, polyethylene glycol, poly(methyl methacrylate), polycarbophil, gelatin, alginate, poly(acrylic acid), polyethylene oxide and chitosan or a derivative thereof.

Surfactant(s) include, but are not limited to be anionic, cationic or non-ionic, selected from but not limited to polysorbate 80 and 20, polyethylene glycol esters, polyethylene glycols, glycerol ethers, glyceryl monoleate, lecithin or mixtures of those compounds.

Co-solvents are selected from, but not limited, to monohydric alcohol or polyhydric alcohols such as polyethylene glycol, polypropyleneglycol and the like.

The composition of the disclosure can further optionally comprise a chelating agent such as disodium salt of EDTA. The composition may comprise suitable water soluble polymers which may be used in the disclosed compositions, including, but not limited to methylcellulose, hydroxyethyl cellulose, hydroxypropyl cellulose, hydroxypropyl methylcellulose, hydroxypropyl ethylcellulose, sodium carboxymethylcellulose, polyvinyl alcohol, polyvinylpyrrolidone, polyethylene glycol, poly(methyl methacrylate), polycarbophil, gelatin, alginate, poly(acrylic acid), polyethylene oxide and chitosan or a derivative thereof.

Stabilizing agents than can be used in the composition disclosed herein include, but are not limited to sodium hydrogen sulfite, glycerin, sodium citrate, butyl hydroxyanisole, benzalkonium chloride, edetic acid and pharmaceutically acceptable salts thereof, tocopherol and derivatives thereof, optionally in combination with sodium edetate.

Isotonizing agents that can be used in the composition include, but are not limited to any specific one like sodium chloride, potassium chloride, ethanol, D-mannitol, glucose, glycerol, glycine, glycerin, dextrose, glyceryl dioleate, xylitol, propylene, and propylene glycol.

The buffering agents that may be used include, but are not limited to, acids, including hydrochloric acid, acetic acid, and citric acid, sodium hydroxide, sodium and potassium salts, including sodium citrate, sodium carbonate, sodium tetraborate, sodium phosphate, sodium acetate, sodium bicarbonate and potassium phosphate.

The preservatives which may be used include but not limited to, sodium bisulfite, edetate disodium sodium bisulfate, sodium thiosulfate, benzalkonium chloride, benzethonium chloride, chlorobutanol, thimerosal, phenylmercuric acetate, benzyl alcohol, i.e., phenylcarbinol, phenylmercuric nitrate, methylparaben, propylparaben, polyvinyl alcohol, phenylethyl alcohol, and Thimerosal.

In some embodiments, thickening agent is methylcellulose, ethyl cellulose, microchrystalline cellulose, hydroxypropyl methylcellulose, hydroxyethylcellulose, sodium carboxymethylcellulose, polyvinyl pyrrolidone, polyvinyl alcohol, sodium chondroitin sulfate, sodium hyaluronate, chitosan; however, the thickening agent is not limited to these polymers.

Permeation enhancers that can be used in compositions disclosed herein include, but are not limited to chitosan, chitosan derivatives, cyclodextrin, bile slats, liposomes, starch microspheres, and glycerrhizin.

Flavor modifiers that can be used in compositions disclosed herein include, but are not limited to menthol, saccharin sodium, sorbitol.

Electrolytes such as sodium chloride and potassium chloride may also be included in the formulation.

Also contemplated herein are the use of commercially available excipients, including, but not limited to Kollidon® 12 PF, Kollidon® 17 PF, Kolliphor® EL, Kolliphor® ELP, and Kolliphor® HS 15 (BASF).

Also contemplated within the scope of embodiments described herein are allopregnanolone formulations that are oil-based suspensions, emulsions, or solutions; or aqueous suspensions, emulsions, or solutions; or combinations thereof. In one instance, such suspensions or emulsions or solutions may further comprise allopregnanolone solubilizing excipients such as one or more cyclodextrins. In another instance, such suspensions or emulsions or solutions may not comprise allopregnanolone solubilizing excipients such as one or more cyclodextrins. In further instances, neat allopregnanolone powder may be insufflated into the nose. Oils that may be used include, but are not limited to canola oil, miglyol oil, peanut oil, and mixtures thereof.

In certain examples, the composition comprising allopregnanolone is administered at a dose of allopregnanolone in the range of about 0.25 mg/kg to about 25 mg/kg. In specific instances, the composition comprising allopregnanolone is administered intranasally at a dose of allopregnanolone in the range of about 0.25 mg/kg to about 25 mg/kg.

In some instances, the composition comprising allopregnanolone is administered 1 to 10 times over a period of 1 to 10 minutes. In specific instances, the composition comprising allopregnanolone is administered intranasally 1 to 5 times in each nostril over a period of 1 to 10 minutes. In some instances, the composition comprising allopregnanolone is administered 1 to 5 times over a period of 1 to 5 minutes, or 1 to 3 minutes.

Determination of an effective amount for administration in a single dosage is well within the capability of those skilled in the art. Generally, an efficacious or effective amount of allopregnanolone is determined by first administering a low dose or small amount of the agent and then incrementally increasing the administered dose or dosages, until a desired effect is observed in the treated subject with minimal or no toxic side effects. Applicable methods for determining an appropriate dose and dosing schedule for administration of a combination of the present invention are described, for example, in Brunton, et al., Goodman and Gilman's The Pharmacological Basis of Therapeutics, 12^{th} Edition, 2010, McGraw-Hill Professional; in a Physicians' Desk Reference (PDR), 66^{th} Edition, 2012; in Loyd, et al., Remington: The Science and Practice of Pharmacy, 22^{nd} Ed., 2012, Pharmaceutical Press; in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, each of which are hereby incorporated herein by reference.

In various embodiments, the compositions are formulated, e.g., for intranasal administration, at a dose in the range of about 5 mg/kg to about 250 mg/kg, about 5 mg/kg to about 200 mg/kg, about 5 mg/kg to about 150 mg/kg, about 5 mg/kg to about 100 mg/kg, about 5 mg/kg to about 50 mg/kg, about 5 mg/kg to about 25 mg/kg, about 5 mg/kg, 10 mg/kg, 13 mg/kg, 15 mg/kg, 16 mg/kg, 20 mg/kg, 25 mg/kg, 30 mg/kg, 35 mg/kg, 40 mg/kg, 45 mg/kg, 50 mg/kg, 75 mg/kg, 100 mg/kg, 125 mg/kg, 150 mg/kg, 200 mg/kg, or 250 mg/kg.

In various embodiments, the allopregnanolone compositions are formulated in unit dosage forms, each dosage containing from about 1-1000 mg, about 2-800 mg, about 5-500 mg, about 10-400 mg, about 50-200 mg, e.g., about 5 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 200 mg, about 300 mg, about 400 mg, about 500 mg, about 600 mg, about 700 mg, about 800 mg, about 900 mg or about 1000 mg of allopregnanolone. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient.

In any of the methods described herein, in a group of embodiments, the subject is a human.

Also provided herein is a method for increasing bioavailability of allopregnanolone in the brain, the method comprising intranasally administering a composition comprising allopregnanolone powder or allopregnanolone in an oil or allopregnanolone aqueous solution with sulfobutylether-β-cyclodextrin or other solubilizing agent as excipient, wherein the concentration of allopregnanolone in certain brain regions is higher after intranasal administration than the concentration in the same brain regions after other modes of administration. Alternatively, the methods described herein provide for higher therapeutic concentrations of allopregnanolone in brain regions relevant to the treatment of the condition susceptible to allopregnanolone treatment and lower concentrations in non-relevant brain regions or in brain regions associated with adverse or toxic effects of allopregnanolone. The methods described herein provide an improvement in comparison with administration of allopregnanolone by other routes, including the intravenous route, where increased concentrations in selective brain regions relevant to the treatment of the disorder are not obtained.

In some embodiments, the methods provided herein allow for selective delivery of allopregnanolone to the forebrain, the methods comprising intranasally administering a composition comprising allopregnanolone and sulfobutylether-β-cyclodextrin, wherein, after intranasally administering, the bioavailability of allopregnanolone in the forebrain is higher compared to bioavailability of allopregnanolone in other regions of the brain. In some embodiments, the methods described herein allow for selective delivery of allopregnanolone to the olfactory bulb and adjacent structures, the methods comprising intranasally administering a composition comprising allopregnanolone and sulfobutylether-β-cyclodextrin, wherein, after intranasally administering, the tissue concentration of allopregnanolone in the olfactory blub and adjacent brain structures is higher compared to the tissue concentration in other regions of the brain or in the whole brain. The olfactory bulb is present in the vertebrate forebrain. Structures adjacent to the olfactory bulb include structures in the forebrain. The forebrain includes the cerebrum (including the frontal lobe, parietal lobe, occipital lobe, and temporal lobe), thalamus, and hypothalamus (part of the limbic system).

The intranasal route of administration potentially bypasses the blood-brain barrier and delivers active agents directly to the brain from the nasal cavity along the olfactory and trigeminal nerves (i.e., “nose-to-brain delivery”). The active agent may be delivered specifically to brain regions relevant to therapeutic activity, resulting in higher concentrations in the relevant brain regions compared with the concentrations in non-relevant brain regions, resulting in improved therapeutic effect and/or improved therapeutic index and/or reduced adverse effects or toxicity. The active agent may undergo endocytosis by olfactory sensory cells, followed by transynaptic axonal transport to olfactory neurons, thereby distributing the drug to other brain regions. Alternatively, the active agent may be transported directly into the cerebral spinal fluid by first passing across the nasal epithelium, then through the perineural space to the subarachnoid space of the brain.

As demonstrated by the Figures and the Examples, intranasally administered allopregnanolone produces therapeutically meaningful blood levels that are maintained for up to 8 hours whereas when allopregnanolone is administered by a conventional parenteral route of administration (e.g., intramuscular administration) therapeutic levels are maintained for about 2 hours. Having therapeutic levels maintained for about 8 hours allows intermittent dosing, on for example a t.i.d. (3 times a day) schedule, eliminating the need for continuous infusion. In addition, intranasal dosing allows self-administration or administration by a lay caregiver. It avoids an invasive and potentially painful mode of administration and eliminates the need for trained medical personnel and confinement to a specialized facility. In some instances, a “therapeutically relevant” plasma level of allopregnanolone is about 50 ng/mL, which is about the target concentration obtained with an FDA-approved dosing scheme for allopregnanolone in the treatment of postpartum depression. It will be understood that the therapeutic levels of allopregnanolone for the treatment of other diseases may be different and other such levels of allopregnanolone are also contemplated within the scope of embodiments presented herein.

As demonstrated by the Figures and the Examples, intranasal allopregnanolone produces persistent (up to 8 hours after dosing) protection in the 6 Hz seizure test, which may reflect the persistent plasma and correspondingly persistent brain levels at therapeutic levels (up to 8 hours after dosing).

It was found that intranasal allopregnanolone (16 mg/kg) was not associated with substantial sedation/general anesthesia as assessed by loss of righting reflex whereas the same dose (16 mg/kg) of allopregnanolone administered intramuscularly produces substantial loss of righting reflex during the period 10 min to 30 min after administration.

Without being bound by any theory, it is believed that this divergence reflects the initial rapid entry and extraction of allopregnanolone into the brain after intramuscular administration, with the allopregnanolone levels in brain then falling at about 30 min due to redistribution into other body compartments (i.e., body fat and muscle). Therefore, intranasal delivery has the additional advantage of avoiding transitory excessively high brain levels that occur with bolus parenteral (intramuscular) administration that may be associated with early adverse effects.

Surprisingly there was no substantial elevation in the seizure threshold as assessed with the timed intravenous PTZ seizure threshold test at 8 hours after intranasal dosing. The PTZ seizure threshold test is a very sensitive test of antiseizure activity. There is therefore a divergence between the effect in the 6 Hz model and the timed intravenous PTZ seizure threshold test. The former model is considered a “limbic” seizure model and may reflect seizure activity in temporal lobe structures that are easily accessed by the olfactory system. In contrast, the timed intravenous PTZ seizure threshold test may assay seizure activity in lower brain structures, including the brainstem. Accordingly, the methods described herein demonstrate a possibility that when allopregnanolone is administered by the intranasal route, allopregnanolone preferentially accesses temporal lobe structures (which may be associated with therapeutic activity) and avoids the remainder of the brain (which may be associated with adverse effects).

It was further found that following intranasal administration, concentrations of allopregnanolone in the olfactory bulb were 10-fold the concentration in the whole brain, demonstrating the occurrence of nose-to-brain delivery.

The active agent may be administered at once, or may be divided into a number of smaller doses to be successively administered at intervals of time, or over an interval of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In some embodiments, the allopregnanolone compositions described herein are administered so as to deliver a first bolus dose; followed by administration of a second/maintenance dosing regimen; wherein, the administration of said second dosing regimen occurs at intervals and lasts for 1-6 days, or longer; and administration of a third downward taper dosing regimen, wherein, the administration of said third downward taper dosing regimen occurs at intervals over 10-100 hours; wherein, collectively, the administrations are provided in sufficient amount to treat a subject suffering from any condition described herein. By way of example only, a bolus dose may be 0.75×, the second/maintenance dosing regimen may comprise doses of 0.5× or lower, and the taper dosing regimen may comprise doses of 0.25× or lower. Optionally, a subject is administered a dose of a composition described herein prophylactically (e.g., prior to onset of symptoms such as, for example, seizures). In other embodiments, the allopregnanolone compositions described herein are administered as multiple doses over a period of minutes to an hour, e.g., 1-5 doses over about 1 to 10 minutes, over about 1 to 5 minutes, over about 1-3 minutes, or over about 1 to 30 minutes or over about 1 to 60 minutes.

In various embodiments, administration of allopregnanolone to a subject results in the prevention or mitigation of one or more symptoms of the disease condition being treated. Symptoms of disease can be compared before and after administration of allopregnanolone to the subject. Administration of allopregnanolone to the subject is considered to be effective if the symptoms no longer occur after administration (e.g., seizures, symptoms of depression), or if the symptoms are reduced, or alleviated after administration.

In various instances, the efficacy is monitored by a caregiver. For example, in a subject experiencing the onset of a seizure or in situations where a seizure has commenced, the subject may require administration of allopregnanolone by a caregiver. If allopregnanolone is administered in an efficacious amount, the seizure, along with the subject's symptoms of the seizure, should terminate or abort. If the seizure does not terminate, a second dose of allopregnanolone can be administered. In a subject experiencing a mood disorder (e.g., anxiety, post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression or treatment resistant depression), a caregiver may monitor the outcome of allopregnanolone treatment based on, e.g., HAM-D scores.

In some instances, the compositions described herein are administered to a subject suffering from any mood order described herein in combination with other anti-depressants (e.g., oral anti-depressants). Examples of such oral anti-depressants suitable for combination therapy include and are not limited to selective serotonin reuptake inhibitors (SSRIs) such as fluoxetine (Prozac®), paroxetine (Paxil®, Pexeva®), sertraline (Zoloft®), citalopram (Celexa®) and escitalopram (Lexapro®); serotonin and norepinephrine reuptake inhibitors (SNRIs) such as duloxetine (Cymbalta®), venlafaxine (Effexor XR®), desvenlafaxine (Pristiq®, Khedezla®) and levomilnacipran (Fetzima®); atypical antidepressants such as trazodone, mirtazapine (Remeron®), vortioxetine (Trintellix®), vilazodone (Viibryd®) and bupropion (Wellbutrin®, Aplenzin®, Forfivo XL®); tricyclic antidepressants such as imipramine (Tofranil®), nortriptyline (Pamelo® r), amitriptyline, doxepin and desipramine (Norpramin®); monoamine oxidase inhibitors (MAOIs) such as tranylcypromine (Parnate®), phenelzine (Nardil®) and isocarboxazid (Marplan®).

Animal models for anxiety and depression are known to the skilled practitioner. For example, certain animal models which may be used include and are not limited to, the Force Swim Test in mice or rats (FST), the Tail Suspension Test in mice, and the Elevated Plus Maze (EPM) and Open Field Test in Mice (OFT). Protocols for such tests are found, for example, in “Animal models for depression-like and anxiety-like behavior,” Joshua Hunsberger, Catharine Duman and Ronald Duman, Protocol Exchange (2007), doi:10.1038/nprot.2007.542, Published online 13 Dec. 2007.

### EXAMPLES

### Example 1

Solutions of allopregnanolone (5α,3α-P) (15 mg/ml) were prepared in 40% sulfobutylether-β-cyclodextrin sodium salt in 0.9% saline. Seizures were induced in mice with pentylenetetrazol (PTZ; 80 g/kg IP). 5α,3α-P solution (6 and 10 mg/kg) or vehicle was administered intranasally 5, 10 and 15 min prior to administration of the PTZ. Animals were observed for 30 min following PTZ. The times to onset of myoclonic body twitches and clonic and tonic seizures were recorded. 5α,3α-P was considered to have antiseizure activity if it delayed the onset of seizure signs in comparison with the time of their occurrence in vehicle-treated animals.

In this maximal PTZ test, 5α,3α-P 6 mg/kg administered intranasally delayed the time to onset of all seizure signs with a pronounced effect on tonic hind limb extension. At 10 mg/kg in addition to a delay in seizure signs, some animals were protected from tonic hind limb extension and mortality. Delay in seizure signs was evidenced when 5α,3α-P was administered 5 min before PTZ but at 15 min it was less active, indicating a short acting effect.

The results demonstrate that 5α,3α-P solubilized with a cyclodextrin excipient exhibits antiseizure activity when administered into the nose. 5α,3α-P acted rapidly to delay seizure onset and improve survival. Delivery by the intranasal route may allow 5α,3α-P to be conveniently and atraumatically administered by a non-medically-trained caregiver to abort ongoing seizures.

### Example 2

Allopregnanolone (16 mg/mL in 40% Captisol® in 0.9% saline) was administered intranasally at a total dose of 16 mg/kg to mice. 2-3 microliters of solution was administered 3-5 times to each nostril (6-10 times total) over a period of 3.5 min.

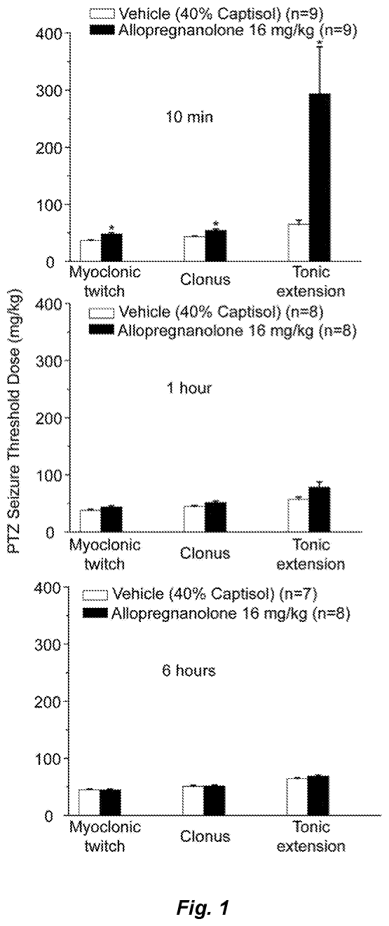

FIG. 1 shows the effect of intranasal allopregnanolone on the seizure threshold in the timed intravenous PTZ seizure threshold test (conducted according to the protocol described by Mandhane, S. M. et al., Seizure (2007) 16, 636-644) at 10 minutes, 1 hour and 8 hours. Ten minutes after intranasal allopregnanolone, there is a large and statistically significant increase in various seizure threshold parameters (including a significant effect on tonic extension) indicating that intranasal allopregnanolone is well absorbed intranasally (i.e., has therapeutically relevant bioavailability).

The effect diminishes by 1 hour and is absent at 8 hours, i.e., only minimal (non-significant) elevation in seizure threshold (including the effect on tonic extension) was observed by 1 hour and/or 8 hours.

### Example 3

Allopregnanolone (16 mg/mL in 40% Captisol® in 0.9% saline) was administered intranasally at a total dose of 16 mg/kg to mice that were mostly <25 g although a few animals weighed up to 28 g. 2-3 microliters of solution was administered 3-5 times to each nostril (6-10 times total) over a period of 3.5 min. Animals were tested in the 6 Hz seizure test (https://panache.ninds.nih.gov/m_6 hztest.aspx), at different intervals following completion of dosing. Groups of 8 animals were tested at each interval. The first group was tested 1 min after the completion of intranasal dosing (i.e., 4.5 min from the start of intranasal dosing).

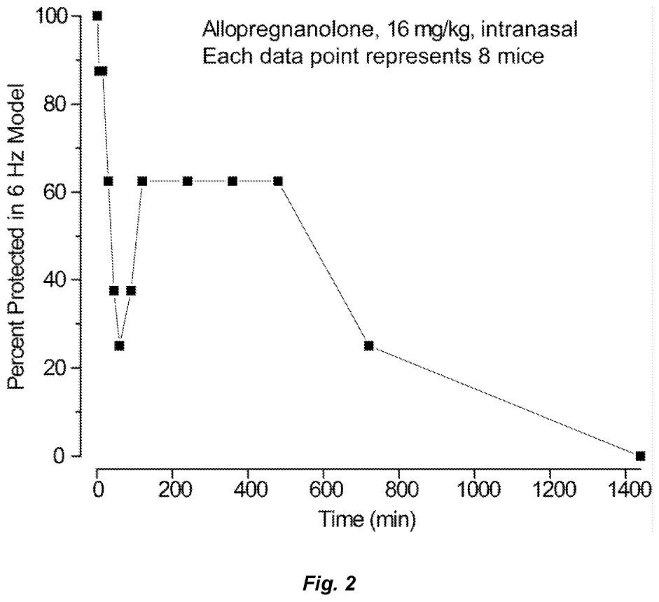

Intranasal dosing of allopregnanolone conferred seizure protection in the 6 Hz seizure test in mice. It was found that dosing of allopregnanolone intranasally over 3.5 min led to an extended period of seizure protection. As shown in FIG. 2, the seizure protection lasted at least 6 hours, and up to about 8 hours after dosing.

The results from Example 2 above contrast with the results of the 6 Hz seizure test of Example 3. In Example 3, there was robust antiseizure activity at 6 hours and antiseizure activity persisted at least up to 8 hours. Thus, unexpectedly intranasal delivery exhibits prolonged effects in the 6 Hz seizure test model compared to the PTZ seizure threshold model. The prolonged seizure protection in the 6 Hz test relates, in part, to prolonged blood levels. However, factors other than maintained blood levels may contribute to the persistent activity in the 6 Hz test with intranasal dosing and the minimal (non-significant) elevation in seizure threshold in the timed intravenous PTZ seizure threshold test. The 6 Hz model is considered a “limbic” seizure model and may reflect seizure activity in temporal lobe structures that are easily accessed by the olfactory system. In contrast, the timed intravenous PTZ seizure threshold test may assay seizure activity in lower brain structures, including the brainstem. When administered by the intranasal route, allopregnanolone possibly accesses forebrain structures, including the temporal lobe (which may be associated with therapeutic activity) and possibly avoids the remainder of the brain (which may be associated with adverse effects).

### Example 4

Allopregnanolone (16 mg/mL in 40% Captisol® in 0.9% saline) was administered intranasally at a total dose of 10 mg/kg. 2-3 microliters of solution was administered 3-5 times to each nostril (6-10 times total) over a period of 3.5 min. Allopregnanolone (6 mg/mL in 24% Captisol® in 0.9% saline) was administered intramuscularly at a total dose of 12 mg/kg. Time course of plasma levels of allopregnanolone following intranasal allopregnanolone (10 mg/kg) and intramuscular allopregnanolone (12 mg/kg) was monitored. At various times after administration, trunk blood was collected in chilled heparinized tubes. In some cases, blood was collected by retro-orbital puncture. Plasma was separated and assayed for allopregnanolone as described in Zolkowska D. et al., Epilepsia 2018 October; 59 Suppl 2:220-227.

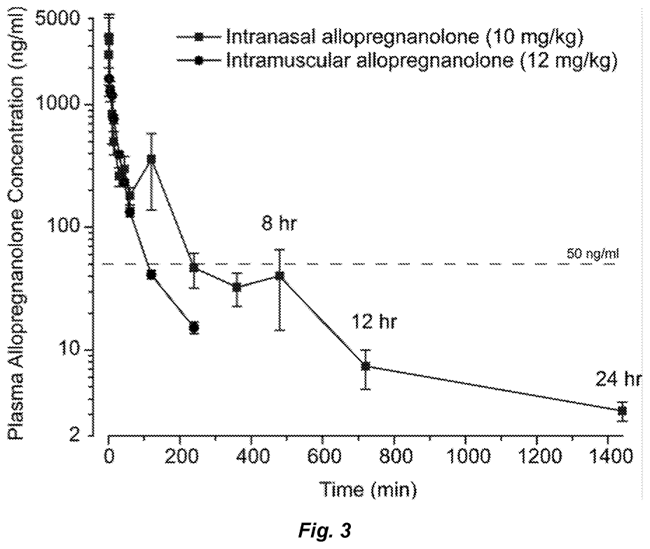

FIG. 3 shows that plasma levels of allopregnanolone with intranasal delivery are sustained for up to 8 hours compared to plasma levels of allopregnanolone with intramuscular delivery which taper off by about 4 hours.

### Example 5

Allopregnanolone (16 mg/mL in 40% Captisol® in 0.9% saline) was administered intranasally at a total dose of 16 mg/kg. 2-3 microliters of solution was administered 3-5 times to each nostril (6-10 times total) over a period of 3.5 min. Allopregnanolone (6 mg/mL in 24% Captisol® in 0.9% saline) was administered intramuscularly at a total dose of 16 mg/kg.

Time course of loss of righting reflex following intranasal allopregnanolone (16 mg/kg) and intramuscular allopregnanolone (16 mg/kg). Loss of righting reflex is a nonnoxious behavioral paradigm that indicates loss of consciousness (hypnosis). Allopregnanolone (16 mg/kg) delivered intramuscularly caused loss of righting reflex in a high proportion of mice during the period 10-30 min whereas the same dose administered intranasally caused loss of righting reflex in only a small proportion of animal up to 15 min after delivery. Intranasal delivery of alloprepregnanolone according to the methods described herein avoids the adverse effects on behavior associated with intramuscular bolus administration.

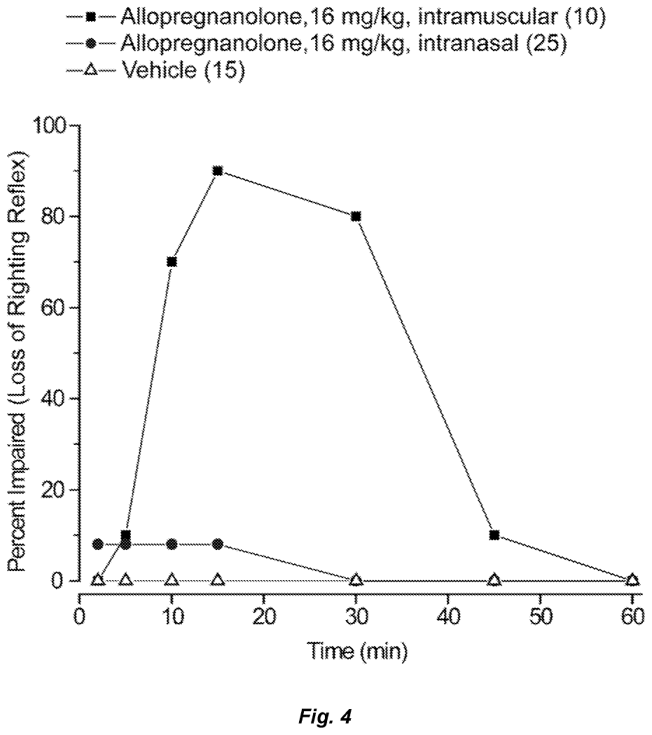

FIG. 4 shows the data for the loss of righting reflex in mice, comparing intranasal and intramuscular administration. Intranasal allopregnanolone (16 mg/kg) was not associated with substantial sedation/general anesthesia as assessed by loss of righting reflex whereas the same dose (16 mg/kg) of allopregnanolone administered intramuscularly produces substantial loss of righting reflex during the period 10 min to 30 min after administration. Accordingly, the intranasal route of administration reduces the occurrence of loss of righting reflex, thereby offering advantages over the intramuscular route of administration.

### Example 6

Allopregnanolone (16 mg/mL in 40% Captisol® in 0.9% saline) was administered intranasally at a total dose of 16 mg/kg. 2-3 microliters of solution was administered 3-5 times to each nostril (6-10 times total) over a period of 3.5 min. Allopregnanolone (6 mg/mL in 24% Captisol® in 0.9% saline) was administered intramuscularly at a total dose of 16 mg/kg.

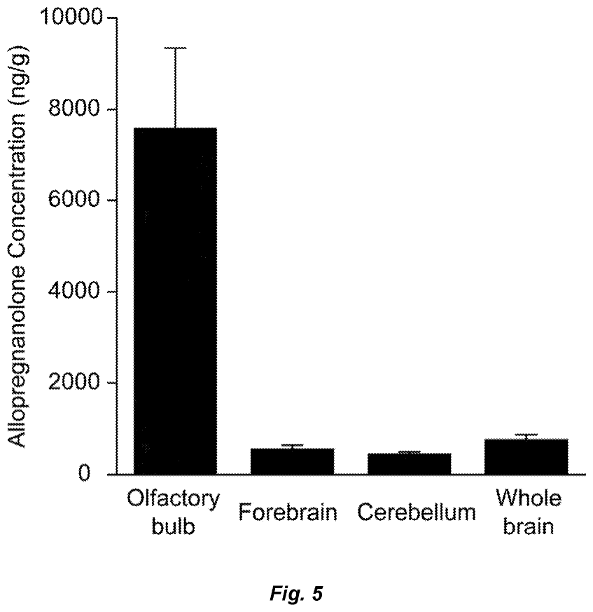

Allopregnanolone (16 mg/kg) or vehicle were administered intranasally to mice. The brains were removed and divided into olfactory bulb, forebrain and cerebellum. The brain sections were weighed, water was added (0.25 mL, 1 mL and 0.5 mL for the olfactory bulb, forebrain and cerebellum, respectively), and the tissue was homogenized. Allopregnanolone was assayed as described in Zolkowska D. et al., Epilepsia 2018 October; 59 Suppl 2:220-227. Bars indicate means±S.E.M. of allopregnanolone concentrations in the brain sections from four mice collected at 1, 2, 5 and 10 min after intranasal dosing. The whole brain allopregnanolone amounts represent the sum of the amounts in the three sections. Levels at the four time points were similar and did not exhibit a consistent trend during the time period sampled justifying averaging. FIG. 5 shows that mean allopregnanolone concentration in the olfactory bulb is 10-fold greater than in the whole brain.

### Example 7

NIH Swiss mice (22-32 g) from Charles River were kept in a vivarium under controlled environmental conditions (22-26° C.; 40-50% humidity) with an artificial 12-h light/dark cycle. Wood chips were used in all cages. Experiments were performed during the light phase of the light/dark cycle after a minimum 30-min period of acclimation to the experimental room. The animal facilities were fully accredited by the Association for Assessment and Accreditation of Laboratory Animal Care. All studies were performed under protocols approved by the Animal Care and Use Committee of the University of California, Davis in strict compliance with the Guide for the Care and Use of Laboratory Animals, Eighth Edition (The National Academies Press, 2011).

### Test Substances and Drug Administration

Allopregnanolone (custom synthesized, >99% pure) was dissolved at a concentration of 16 mg/ml in an aqueous solution containing 0.9% NaCl and 40% sulfobutylether-β-cyclodextrin (Captisol®; Ligand Pharmaceuticals, San Diego, Calif.). The allopregnanolone solution was instilled in the nose using a series of small volume drops over the course of 3.5 min to reach a total dose 10 or 16 mg/kg. Pharmaceutical grade midazolam and diazepam were purchased from Hospira (Lake Forest, Ill.); the solution concentrations were 1 mg/ml and 5 mg/ml, respectively. Midazolam and diazepam were administered intransally at a dose of 1 mg/kg using the same procedure as allopregnanolone. Vehicle solutions were prepared using the excipients as in the commercial products. The vehicle for midazolam solution contained 0.8% NaCl, 0.01% EDTA disodium salt, and 1% benzyl alcohol adjusted to pH 3.3 to 3.5. The vehicle for diazepam solution contained 40% propylene glycol and 10% ethyl alcohol. Pentylenetetrazol (PTZ) was purchased from Sigma-Aldrich and was used as a 10 mg/ml solution in 0.9% NaCl.

### Timed Intravenous Seizure Threshold Test in Mice

The thresholds for various behavioral seizure stages induced by the PTZ and bicuculine were determined by infusing the convulsant drugs via a 27-gauge, 0.75-inch “butterfly” needle inserted into the lateral tail vein. PTZ (10 mg/ml) and bicuculline (0.1 mg/ml) were infused IV at a constant rate of 0.5 ml/min using a 5-ml syringe (BD Biosciences, Franklin Lakes, N.J.) mounted on an infusion pump (Model ‘11’ Plus Syringe Pump; Harvard Apparatus, Holliston, Mass.). The syringe was connected to the needle by polyethylene tubing. The infusion was stopped at 3 min or at the onset of tonic extension, whichever occurred first. The time to occurrence (“infusion duration”) to the following endpoints from the start of the convulsant infusion were determined: (1) the first myoclonic jerk; (2) the onset of generalized clonus; and (3) the onset of tonic extension. Infusion duration values represent the time from the start of the convulsant infusion to the onset of the endpoint. The threshold value (mg/kg) for each endpoint was determined according to the following formula: (infusion duration [s]×infusion rate [ml/min]×convulant concentration [10 mg/ml or 0.1 mg/ml]×1000)/(60 s×body weight of mouse [g]).

### Maximal PTZ Seizure Test

Mice were injected intraperitoneally with PTZ (80 mg/kg) and were observed for a 30-min period. The times to occurrence of the 3 seizure signs as in the timed intravenous PTZ seizure test were recorded. Allopregnanolone (10 mg/kg) or vehicle was administered intranasally 10 min prior to the PTZ injection.

### Horizontal Screen Test

At various times after intranasal administration, mice were placed on a horizontally oriented grid (consisting of parallel 1.5-mm diameter rods situated 1 cm apart) for 2-3 s and the grid was inverted and held approximately 30-40 cm above a padded surface for a maximum of 60 s. If the animal fell from the grid, the latency from the time of inversion to the time of fall was recorded. For animals that did not fall from the grid, the holding time was recorded as 60 s.

### Loss of Righting Reflex Test

For the test, the mouse was placed on its back on a tabletop. Animals that failed to right themself through 180° are scored as positive.

### Blood and Brain Pharmacokinetic Analyses

At various times after intranasal administration, trunk blood samples were collected into chilled tubes. Brains were removed, separated into 3 sections (olfactory bulb, forebrain with midbrain, cerebellum with attached pons and medulla) and fast-frozen on dry ice. Blood was centrifuged at 1500 RCF for 10 min to separate the plasma. Brain and plasma samples were stored at −80° C. until processing. Plasma and brain samples were analyzed for allopregnanolone as described in Zolkowska et al (2018). The lower limit of quantification in plasma is 2.5 ng/ml.

### Example 8

### Comparison of Intranasal Allopregnanolone, Midazolam and Diazepam on Seizure Threshold

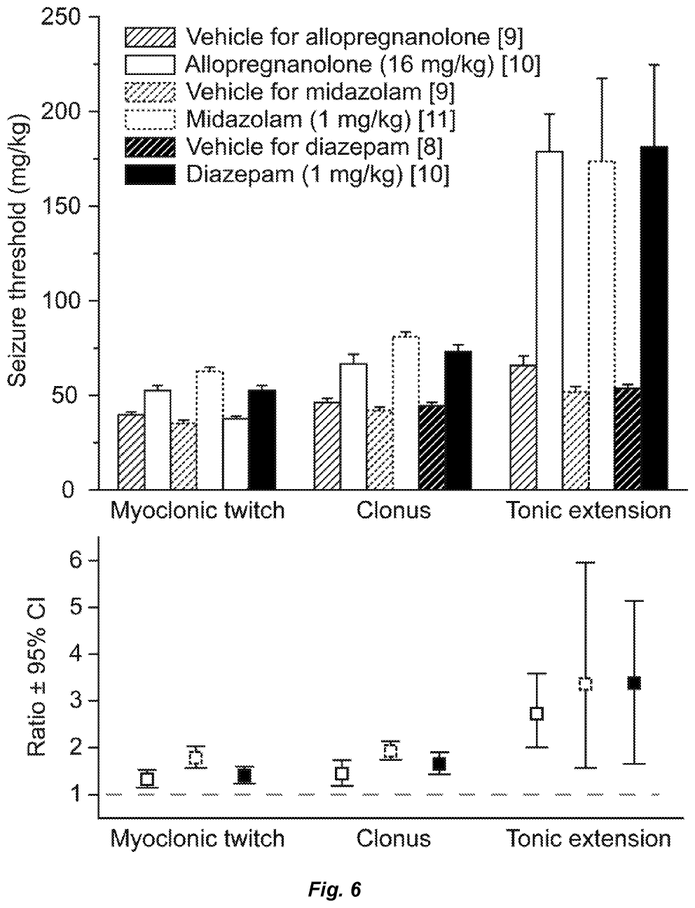

The effects of intranasal allopregnanolone at a dose of 16 mg/kg on the thresholds for myoclonic twitch, clonus and tonic extension were assessed in the timed intravenous PTZ threshold test. As shown in FIG. 6, the thresholds for all seizure endpoints were increased compared to vehicle 10 min following the completion of nasal administration. The extent of threshold elevation was similar for the myoclonic twitch and clonus endpoints and higher for the tonic extension endpoint (ratio of mean threshold values in allopregnanolone and vehicle groups, 1.3, 1.4, and 2.7, respectively). For comparison, we also studied midazolam and diazepam. At a dose of 1 mg/kg, the two benzodiazepines produced comparable elevations in the thresholds to the three seizure endpoints.

### Time Course of Intranasal Allopregnanolone Effects on Tonic Extension Endpoint

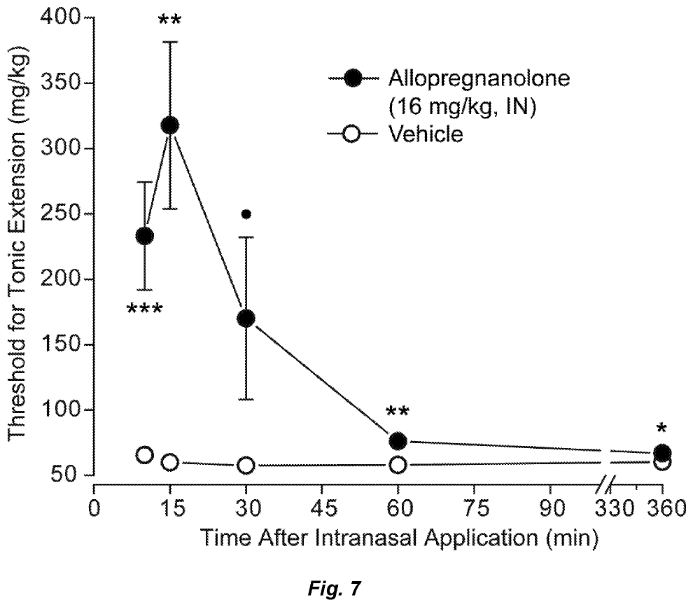

Separate groups of animals were evaluated in the timed intravenous PTZ seizure threshold test with intervals between intranasal dosing of allopregnanolone (16 mg/kg) and vehicle of 10, 15, 30, 60, and 360 min. As shown in FIG. 7, a statistically significant increase in threshold was obtained at the earliest (10 min) allopregnanolone group compared with the corresponding vehicle group. The maximum elevation occurred at 15 min (ratio of mean thresholds in allopregnanolone and vehicle groups, 5.3; 95% CI: 3.1-7.7) and only a small elevation was obtained at 60 min (ratio, 1.3; 95% CI: 1.1-1.5). At 360 min there continued to be a statistically significant elevation in threshold but the magnitude was very small (ratio, 1.1; 95% CI: 1.0-1.2).

### Effects of Intranasal Allopregnanolone in the Maximal PTZ Seizure Test

As an alternative to the PTZ threshold test, we assessed the effect of pretreatment with intranasal allopregnanolone in the maximal PTZ seizure test. All 30 vehicle-treated animals receiving a maximal dose of PTZ exhibited a sequence of myoclonic twitch, clonus and tonic extension with the following mean±S.E.M. latencies: 63.9±1.7 s, 76.2±2.5 s, and 204.1±17.1 s. Intransal allopregnanolone (10 mg/kg) treatment administered 10 min before PTZ increased the latencies to the onset of the 3 seizure signs. The ratios of the latencies in the allopregnanolone group to the vehicle group were as follows: myoclonic twitch, 1.3 (95% CI: 1.2-1.5); clonus, 1.5 (95% CI: 1.2-1.8); tonic extension, 4.4 (95% CI: 2.4-6.6). All of the allopregnanolone treated animals exhibited myoclonic twitch and clonus. However, of the 14 animals tested, 5 failed to exhibit tonic extension; the latency was taken to be the entire 30 min (1800 s) observation period.

### Effects of Intranasal Allopregnanolone in the Intravenous Bicuculline Threshold Test

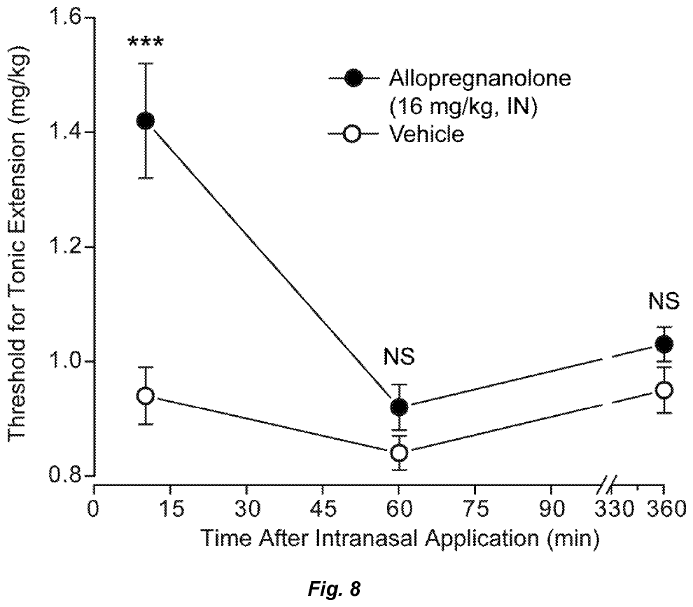

As shown in FIG. 8, intranasal allopregnanolone (16 mg/kg) caused an elevation in the threshold for tonic extension in the 10 min group but not in the groups tested at later times. At the 10 min time point, the ratio of the threshold in the bicuculline group to the threshold in the corresponding vehicle group is 1.5 (95% CI: 1.2-1.8).

### Comparison of Intranasal Allopregnanolone, Midazolam and Diazepam on Motor Impairment

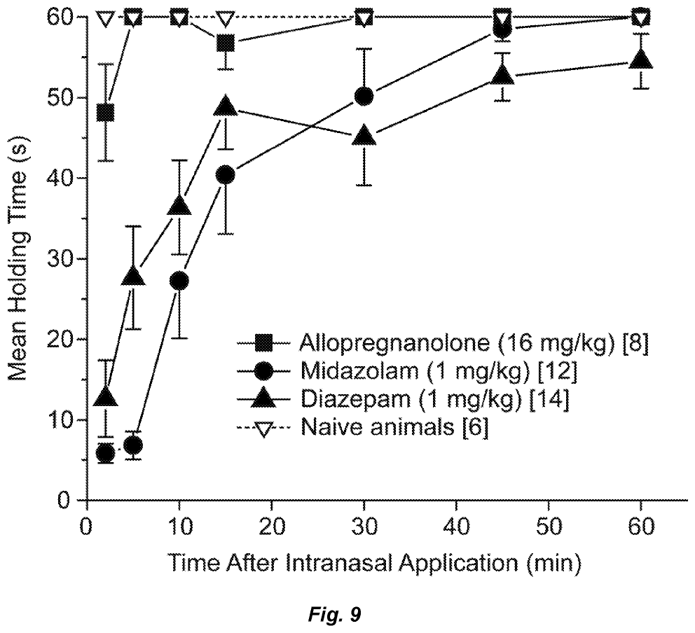

The horizontal screen test was used to assess motor impairment following intranasal administration of the test substances (FIG. 9). Naive animals that did not receive a test substance did not fall from the grid. Similarly, most (5 of 8) animals receiving intranasal allopregnanolone (16 mg/kg) did not fall from the grid; the remainder fell only at the 2 min time point and 1 at the 15 min time point. By contrast all midazolam and diazepam animals fell from the grid. Comparisons of the groups were made by calculating the area under the mean holding time versus time after nasal administration curves for each animal. As noted in the caption to FIG. 9, the curves for untreated (naive) animals and allopregnanolone were not significantly different by this measure. Similarly, the curves for the midazolam and diazepam groups were not significantly different. In contrast, comparisons between the allopregnanolone and midazolam, and allopregnanolone and diazepam groups were significantly different.

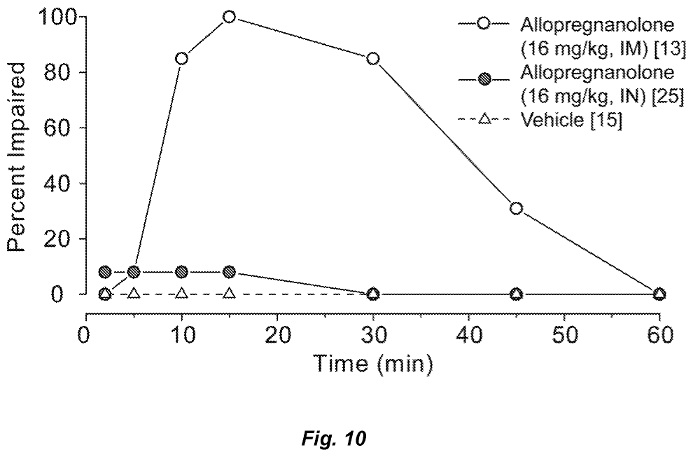

Another means of assessing the propensity of intranasal allopregnanolone to cause neurological impairment (motor impairment, sedation or general anesthesia) is with the loss of righting reflex test. As shown in FIG. 10, 16 mg/kg intranasal allopregnanolone had low propensity to impair neurological function significantly as only 2 of 25 animals exhibited loss of righting reflex during a 60 min observation period after dosing. By contrast, 9 of 10 animal receiving the same dose of allopregnanolone intramuscularly exhibited loss of righting reflex.

### Pharmacokinetics of Allopregnanolone in Plasma and Brain Following Intranasal Delivery

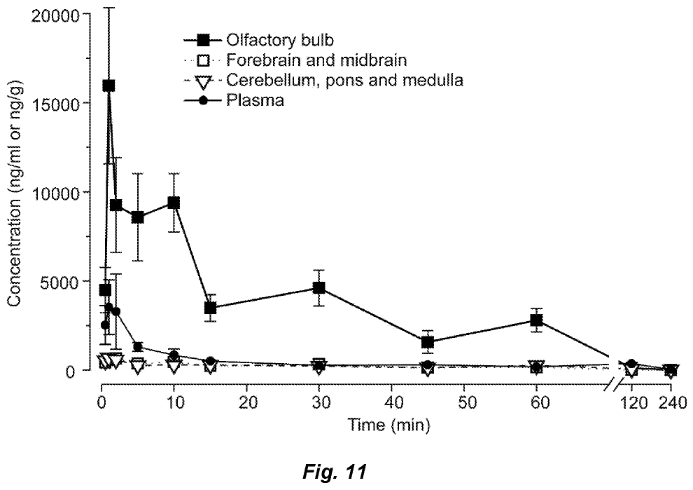

FIG. 11 shows the results of pharmacokinetic measurements of allopregnanolone in plasma and in various brain regions following intranasal dosing with 10 mg/kg allopregnanolone. The plasma Cₘₐₓ value is 3500 ng/ml with Tₘₐₓ at 1 min; fitting the overall curve gave a t_{1/2} value of 49 min, with an initial phase t_{1/2} value of 4.9 min during the first 15 min. Although the nominal Tₘₐₓ was at 1 min, the time measurements are with respect to the end of the 3.5 min slow instillation of allopregnanolone into the nose. Therefore, the true Cₘₐₓ is not known with precision but is likely <5 min. It is noteworthy that the olfactory bulb concentrations are relatively very high in relation to the plasma levels and the concentrations in other brain regions. The olfactory bulb Cₘₐₓ value is 15,960 ng/g with Tₘₐₓ at 1 min. The other two brain sections achieved lower levels with Cₘₐₓ values of 600-700 ng/g.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating or preventing a condition susceptible to allopregnanolone selected from the group consisting of traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, and smoking cessation, in a subject in need thereof, comprising administering to the subject intranasally, buccally, sublingually, lingually or transdermally, a composition comprising allopregnanolone and a pharmaceutically acceptable excipient suitable for solubilizing allopregnanolone.

2. The method of claim 1, wherein the pharmaceutically acceptable excipient suitable for solubilizing allopregnanolone is β-cyclodextrin, hydroxypropyl-β-cyclodextrin or sulfobutylether-β-cyclodextrin, or a combination thereof.

3. A method of treating or preventing a condition susceptible to allopregnanolone selected from the group consisting of traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, and smoking cessation, in a subject in need thereof, comprising administering to the subject an intranasal composition comprising allopregnanolone and sulfobutylether-β-cyclodextrin.

4. The method of claim 1, wherein the condition susceptible to allopregnanolone is anxiety, post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD) or secondary depression.

5. The method of claim 1, wherein the condition susceptible to allopregnanolone is postpartum depression (PPD).

6. The method of claim 1, wherein the condition susceptible to allopregnanolone is post-traumatic stress disorder (PTSD).

7. The method of claim 1, wherein the condition susceptible to allopregnanolone is major depressive disorder (MDD).

8. The method of claim 1, wherein the condition susceptible to allopregnanolone is secondary depression.

9. The method of claim 8, wherein the depression is secondary to a thyroid gland disorder, cancer, cardiovascular disease, or prolonged pain.

10. The method of claim 1, wherein the condition susceptible to allopregnanolone is epilepsy or seizures.

11. The method of claim 10, wherein the seizures are acute seizures, tonic seizures, clonic seizures, or seizure clusters.

12. The method of claim 10, wherein the epilepsy is status epilepticus or myoclonic epilepsy.

13. The method of claim 1, wherein the sulfobutylether-β-cyclodextrin is sulfobutylether-β-cyclodextrin sodium salt.

14. The method of claim 1, wherein the composition comprising allopregnanolone comprises about 10-25 mg/mL of allopregnanolone in a solution of sulfobutylether-β-cyclodextrin sodium salt in saline.

15. The method of claim 14, wherein the solution of sulfobutylether-β-cyclodextrin sodium salt in saline comprises about 10-60% sulfobutylether-β-cyclodextrin sodium salt in saline.

16. The method of claim 14, wherein the solution of sulfobutylether-β-cyclodextrin sodium salt in saline comprises about 40% sulfobutylether-β-cyclodextrin sodium salt in saline.

17. The method of claim 16, wherein the saline is 0.9% saline.

18. The method of claim 1, wherein the composition comprising allopregnanolone is an intranasal composition that comprises 16 mg/mL allopregnanolone in 40% sulfobutylether-β-cyclodextrin in 0.9% saline.

19. The method of claim 1, wherein the composition comprising allopregnanolone is administered at a dose of allopregnanolone in the range of about 0.25 mg/kg to about 25 mg/kg.

20. The method of claim 1, wherein the composition comprising allopregnanolone is administered intranasally at a dose of allopregnanolone in the range of about 0.25 mg/kg to about 25 mg/kg.

21. The method of claim 1, wherein the composition comprising allopregnanolone is administered 1 to 10 times over a period of 1 to 10 minutes.

22. The method of claim 1, wherein the composition comprising allopregnanolone is administered intranasally 1 to 5 times over a period of 1 to 10 minutes.

23. The method of claim 1, wherein the subject is a human.

24. A method for selective delivery of allopregnanolone to the olfactory bulb and adjacent structures, the method comprising intranasally administering a composition comprising allopregnanolone and sulfobutylether-β-cyclodextrin, wherein, after intranasally administering, the tissue concentration of allopregnanolone in the olfactory blub and adjacent brain structures is higher compared to the tissue concentration in other regions of the brain or in the whole brain.
